(19) United States
(12) Patent Application Publication
(10) Pub. No.: US 2010/0323028 A1
(43) Pub. Date: Dec. 23, 2010

(54) METHOD AND COMPOSITION FOR TREATING PULMONARY HEMORRHAGE

(75) Inventors: Stephen Kent Webel, Baylis, IL (US); Lindsey Wilson, Jamestown, IN (US)

Correspondence Address: BARNES & THORNBURG LLP, 11 SOUTH MERIDIAN, INDIANAPOLIS, IN, 46204, US

(73) Assignee: JBS UNITED, INC., Sheridan, IN (US)

(21) Appl. No.: 12/745,398

(22) PCT Filed: Nov. 25, 2008
(86) PCT No.: PCT/US08/84697

§ 371 Date: May 28, 2010

Related U.S. Application Data
(60) Provisional application No. 60/991,436, filed on Nov. 30, 2007.

(51) Int. Cl. A61K 35/60; A61K 31/202; A61K 31/232; A61K 36/02; A61P 9/00

(52) U.S. Cl. 424/523; 514/560; 514/552; 424/195.17

(57) ABSTRACT

The invention relates to methods and compositions for treating and preventing pulmonary hemorrhage in an animal, and for reducing red blood cells in the airways of an animal. More particularly, the invention relates to methods and compositions for treating and preventing pulmonary hemorrhage in an animal, and for reducing red blood cells in the airways of an animal wherein a feed composition comprising an about 3:1 to an about 1:3 ratio of docosahexaneoic acid to eicosapentaenoic acid is fed to the animal.

### CROSS REFERENCE TO RELATED APPLICATION

This application claims the benefit under 35 U.S.C. §119(e) of U.S. Provisional Application Ser. No. 60/991,436, filed on Nov. 30, 2007, incorporated herein by reference in its entirety.

### FIELD OF THE INVENTION

The invention relates to methods and compositions for treating and preventing pulmonary hemorrhage in an animal, and for reducing red blood cells in the airways of an animal. More particularly, the invention relates to methods and compositions for treating and preventing pulmonary hemorrhage in an animal, and for reducing red blood cells in the airways of an animal wherein a feed composition comprising an about 3:1 to an about 1:3 ratio of docosahexanenoic acid to eicosapentaenoic acid is fed to the animal.

### BACKGROUND AND SUMMARY

Exercise-induced pulmonary hemorrhage (EIPH) is a serious and widespread condition in horses competing in racing and other high-performance equestrian events. EIPH causes pulmonary hemorrhage in horses during or following strenuous exercise. In the most severe cases, blood is observed in the nostrils of the animal. However, in most cases, examination with a bronchoscope is required to detect and quantify pulmonary bleeding. Examination of bronchoalveolar lavage fluid (BAL) is one means for detecting the presence of red blood cells in an animal susceptible to EIPH. Generally, examination of thoroughbred racehorses within two hours following a race shows an incidence of EIPH in 42 to 90% of the animals. EIPH is associated with poor performance, leading to decreased lung function over time. A variety of treatments have been used for EIPH, but only FLAIR™ nasal strip and furosemide (Lasix and Salix) have been shown to be effective in reducing EIPH. However, the two treatments alone or in combination have not been able to totally eliminate EIPH. In addition furosemide is not allowed for all performance horses.

Omega-3 and omega-6 fatty acids and their metabolites regulate numerous activities in vivo, including disease resistance, platelet function and vessel wall contractions. Moreover, supplementation of omega-3 fatty acids and/or gamma-linolenic acid present in the diet of animals and humans are reported to have favorable effects on heart disease, autoimmune disorders, diabetes, renal disease, cancer, and immunity as well as learning, visual acuity and neurological function. On a cellular level long chain omega-3 fatty acids can be incorporated into the phospholipid fraction of cell membranes where they influence membrane permeability/fluidity and transport. This represents a storage form of these fatty acids, where they remain until acted upon by phospholipase enzymes which release them for further conversion to eicosanoids.

Linoleic and alpha-linolenic acids are C₁₈-containing fatty acids that are parent compounds of the omega-6 and omega-3 families of fatty acids, respectively. Omega-3 and omega-6 fatty acids undergo unsaturation (i.e., adding double bonds) and sequential elongation from the carboxyl end (i.e., adding 2-carbon units) with the D6-desaturase enzyme being the rate limiting enzyme in metabolism of these long chain fatty acids. The same enzymes are used for these families, making the families antagonistic to one another. Such antagonism, resulting from requirements for the same enzymes, extends into the further metabolism of the C₂₀-containing members of these families into metabolites called eicosanoids.

The polyunsaturated fatty acids, including omega-3 and omega-6 fatty acids, differ from the other fatty acids in that they cannot be synthesized in the body from saturated or monounsaturated fatty acids, but must be obtained in the diet. The omega-6 fatty acid, linoleic acid, is found in high quantities in vegetable oils such as corn, cottonseed, soybean, safflower and sunflower oil. The omega-3 fatty acid, alpha-linolenic acid, is found in high quantities in flaxseed oil, linseed oil, perilla oil and canola oil. Other important compounds include arachidonic acid, found in animal fat; gamma-linolenic acid, found in evening primrose oil, borage oil, and blackcurrant oil; and eicosapentaenoic acid, docosahexaenoic acid, and docosapentaenoic acid derived from fish, algal, and plant sources. These long-chain fatty acids can be formed in the body by elongation and desaturation of the parent linoleic and alpha-linolenic acids if the parent compounds are supplied in the diet.

Applicants have discovered that supplementation of the diet of animals with polyunsaturated fatty acids, including omega-3 fatty acids, can be used to prevent pulmonary hemorrhage in the animal. Furthermore, supplementation of the diet of animals with omega-3 fatty acids is effective in treating an animal known to suffer from pulmonary hemorrhage, for example, by decreasing the number of red blood cells present in the airways of the animal.

In one embodiment, a method of preventing pulmonary hemorrhage in an animal is provided. The method comprises the steps of administering to said animal an effective amount of a feed composition comprising omega-3 fatty acids or esters thereof to prevent pulmonary hemorrhage in the animal, wherein the omega-3 fatty acids in the final feed composition comprise docosahexaenoic acid and eicosapentaenoic acid, and wherein the docosahexaenoic acid to eicosapentaenoic acid ratio in the final feed composition is about 3:1 to about 1:3. In various aspects of this method embodiment, the source of omega-3 fatty acids or esters thereof is selected from the group consisting of an algal composition, a fish composition, and combinations thereof, the algal composition is in the form of algal products selected from the group consisting of algal-derived oils, algal-derived gels, algal-derived pastes, algal-derived dried products, and derivatives thereof, the algal composition comprises algal products selected from the group consisting of whole algal cell products, ground algal products, and residual products, the fish composition is selected from the group consisting of a fish oil, a fish meal product, and combinations thereof, the fish composition comprises a fish oil from a North Atlantic cold water fish, the fish oil comprises an oil selected from the group consisting of menhaden oil, salmon oil, and haddock oil, the omega-3 fatty acids comprise C₂₂ omega-3 fatty acids, the omega-3 fatty acids comprise C₂₀ omega-3 fatty acids, the feed composition further comprises omega-6 fatty acids or esters thereof, the feed composition is administered daily to the animal, the feed composition as a final mixture further comprises an antioxidant, the docosahexaenoic acid to eicosapentaenoic acid ratio in the final feed composition is about 2:1, the amount of the docosahexaenoic acid fed to said animal is at least 10 grams/day, the amount of the eicosapentaenoic acid fed to said animal is at least 5 grams/day, the pulmonary hemorrhage is exercise-induced pulmonary hemorrhage, and/or the animal is selected from the group consisting of a horse, a greyhound, and a racing camel.

In another illustrative aspect, a method of treating pulmonary hemorrhage in an animal is provided. The method comprises the step of administering to the animal an effective amount of a feed composition comprising omega-3 fatty acids or esters thereof to treat pulmonary hemorrhage in said animal, wherein the omega-3 fatty acids in the final feed composition comprise docosahexaenoic acid and eicosapentaenoic acid, and wherein the docosahexaenoic acid to eicosapentaenoic acid ratio in the final feed composition is about 3:1 to about 1:3. In various aspects of this method embodiment, the source of omega-3 fatty acids or esters thereof is selected from the group consisting of an algal composition, a fish composition, and combinations thereof, the algal composition is in the form of algal products selected from the group consisting of algal-derived oils, algal-derived gels, algal-derived pastes, algal-derived dried products, and derivatives thereof, the algal composition comprises algal products selected from the group consisting of whole algal cell products, ground algal products, and residual products, the fish composition is selected from the group consisting of a fish oil, a fish meal product, and combinations thereof, the fish composition comprises a fish oil from a North Atlantic cold water fish, the fish oil comprises an oil selected from the group consisting of menhaden oil, salmon oil, and haddock oil, the omega-3 fatty acids comprise C₂₂ omega-3 fatty acids, the omega-3 fatty acids comprise C₂₀ omega-3 fatty acids, the feed composition further comprises omega-6 fatty acids or esters thereof, the feed composition is administered daily to the animal, the feed composition as a final mixture further comprises an antioxidant, the docosahexaenoic acid to eicosapentaenoic acid ratio in the final feed composition is about 2:1, the amount of the docosahexaenoic acid fed to said animal is at least 10 grams/day, the amount of the eicosapentaenoic acid fed to said animal is at least 5 grams/day, the pulmonary hemorrhage is exercise-induced pulmonary hemorrhage, and/or the animal is selected from the group consisting of a horse, a greyhound, and a racing camel.

In yet another embodiment, a method of decreasing the red blood cell count in the airways of an animal is provided. The method comprises the step of administering to the animal an effective amount of a feed composition comprising omega-3 fatty acids or esters thereof to decrease the red blood cell count in the airways of the animal, wherein the omega-3 fatty acids in the final feed composition comprise docosahexaenoic acid and eicosapentaenoic acid, and wherein the docosahexaenoic acid to eicosapentaenoic acid ratio in the final feed composition is about 3:1 to about 1:3. In various aspects of this method embodiment, the source of omega-3 fatty acids or esters thereof is selected from the group consisting of an algal composition, a fish composition, and combinations thereof, the algal composition is in the form of algal products selected from the group consisting of algal-derived oils, algal-derived gels, algal-derived pastes, algal-derived dried products, and derivatives thereof, the algal composition comprises algal products selected from the group consisting of whole algal cell products, ground algal products, and residual products, the fish composition is selected from the group consisting of a fish oil, a fish meal product, and combinations thereof, the fish composition comprises a fish oil from a North Atlantic cold water fish, the fish oil comprises an oil selected from the group consisting of menhaden oil, salmon oil, and haddock oil, the omega-3 fatty acids comprise C₂₂ omega-3 fatty acids, the omega-3 fatty acids comprise C₂₀ omega-3 fatty acids, the feed composition further comprises omega-6 fatty acids or esters thereof, the feed composition is administered daily to the animal, the feed composition as a final mixture further comprises an antioxidant, the docosahexaenoic acid to eicosapentaenoic acid ratio in the final feed composition is about 2:1, the amount of the docosahexaenoic acid fed to said animal is at least 10 grams/day, the amount of the eicosapentaenoic acid fed to said animal is at least 5 grams/day, the pulmonary hemorrhage is exercise-induced pulmonary hemorrhage, and/or the animal is selected from the group consisting of a horse, a greyhound, and a racing camel.

### BRIEF DESCRIPTION OF THE DRAWINGS

FIG. 1. shows red blood cell counts obtained from lavage fluid.

FIG. 2. shows raw data for lavage red blood cells.

FIG. 3. shows lavage fluid cells expressing phagocytosis and oxidative burst.

FIG. 4. shows serum eicosapentaenoic, docosahexaenoic, alpha linolenic, and arachidonic acid levels in horses fed diets enriched in omega-3 fatty acids or horses fed control diets. Serum was collected after an average of 30, 82.9, and 145.4 days of feeding.

FIG. 5. shows white blood cell counts obtained from lavage fluid.

FIG. 6. shows raw data for lavage white blood cells.

### DETAILED DESCRIPTION OF THE INVENTION

While the invention is susceptible to various modifications and alternative forms, specific embodiments will herein be described in detail. It should be understood, however, that there is no intent to limit the invention to the particular forms described, but on the contrary, the intention is to cover all modifications, equivalents, and alternatives falling within the spirit and scope of the invention.

Methods for preventing pulmonary hemorrhage in an animal are described, wherein a feed composition comprising omega-3 fatty acids or esters thereof is administered to the animal. Additionally, methods for treating pulmonary hemorrhage in an animal are described, wherein a feed composition comprising omega-3 fatty acids or esters thereof is administered to the animal. In another embodiment, methods are described for decreasing the red blood cell count in the airways of an animal by administering to the animal a feed composition comprising omega-3 fatty acids or esters thereof.

In one embodiment, a method of preventing pulmonary hemorrhage in an animal is described. The method comprises the step of administering to the animal an effective amount of a feed composition comprising omega-3 fatty acids or esters thereof to prevent pulmonary hemorrhage in the animal. In the method described, the omega-3 fatty acids in the final feed composition comprise docosahexaenoic acid and eicosapentaenoic acid, and the docosahexaenoic acid to eicosapentaenoic acid ratio in the final feed composition is about 3:1 to about 1:3.

In another embodiment, a method of treating pulmonary hemorrhage in an animal is described. The method comprises the step of administering to the animal an effective amount of a feed composition comprising omega-3 fatty acids or esters thereof to treat pulmonary hemorrhage in the animal. In the method described, the omega-3 fatty acids in the final feed composition comprise docosahexaenoic acid and eicosapentaenoic acid, and docosahexaenoic acid to eicosapentaenoic acid ratio in the final feed composition is about 3:1 to about 1:3.

In yet another embodiment, a method of decreasing the red blood cell count in the airways of an animal is described. The method comprises the step of administering to the animal an effective amount of a feed composition comprising omega-3 fatty acids or esters thereof to decrease the red blood cell count in the airways of the animal. In the method described, the omega-3 fatty acids in the final feed composition comprise docosahexaenoic acid and eicosapentaenoic acid, and the docosahexaenoic acid to eicosapentaenoic acid ratio in the final feed composition is about 3:1 to about 1:3.

The “airways” of an animal as used herein include those parts of the respiratory system through which air flows, to get from the external environment to the alveoli, including but not limited to, the mouth, nose, nasal cavity, throat, trachea, larynx, pharynx, lungs, bronchi, bronchioles, alveoli, etc.

The compositions described herein contain, in particular, a source of omega-3 fatty acids or esters thereof; such as products from an algal source (e.g., algal oils, dried algal products, and residuals and derivatives thereof), fish sources (e.g., fish oils, fish meal products, and oils derived from fish meal), or combinations thereof. The algal and fish products serve as a source of omega-3 fatty acids/esters, such as eicosapentaenoic acid (EPA), docosahexaenoic acid (DHA), and docosapentaenoic acid (DPA), or mixtures thereof.

Any source of omega-3 fatty acids may be used in the methods and compositions described herein. In one embodiment, omega-3 fatty acid sources useful in the methods and compositions described comprise C₂₂ omega-3 fatty acids and/or C₂₀ omega-3 fatty acids. In another embodiment, the feed composition as a final mixture will have a DHA:EPA ratio of about 3:1 to about 1:3. In another illustrative embodiment, the feed composition as a final mixture comprises DHA and EPA in a DHA:EPA ratio of about 2:1 to about 1:3. In various illustrative embodiments, the final feed compositions as described herein comprise DHA and EPA in a ratio of about 3:1 to about 2:1, about 3:1 to about 1:1, about 3:1 to about 1:2, or about 2:1 to about 1:2.

In various illustrative aspects, the final feed composition comprises DHA and EPA in a DHA:EPA ratio of about 3:1, about 2.5:1, about 2:1, about 1.5:1, about 1:1, about 1:1.5, about 1:2, about 1:2.5, or about 1:3. In one illustrative embodiment, the amount of the docosahexaenoic acid fed to the animal is at least 10 grams/day. In another illustrative embodiment, the amount of the eicosapentaenoic acid fed to the animal is at least 5 grams/day.

Fatty acids with no double bonds are termed saturated fatty acids, those with one double bond are termed monounsaturated fatty acids, and those with multiple double bonds are termed polyunsaturated fatty acids. Overall digestibility appears to increase with the degree of unsaturation. A convenient shorthand system is used in this specification to denote the structure of fatty acids. This system uses a number denoting the number of carbons in the hydrocarbon chain, followed by a colon and a number indicating the number of double bonds in the molecule, and then by a “w6” or a “w3” to denote “omega-6” or “omega-3”, respectively (e.g., 22:5w6). The “w6” or a “w3” denotes the location of the first double bond from the methyl end of the fatty acid molecule. Trivial names in the w6 series of fatty acids include linoleic acid (18:2w6), gamma-linoleic acid (18:3w6), and arachidonic acid (20:4w6). The only fatty acid in the w3 series with a trivial name is alpha-linolenic acid (18:3w3). For the purposes of this application a fatty acid with the nomenclature 20:5w3 is eicosapentaenoic acid, with the nomenclature 22:6w3 is docosahexaneoic acid, and with the nomenclature 22:5w3 is docosapentaenoic acid.

The methods of the present invention utilize an omega-3 fatty acid-containing composition as a source of long chain omega-3 fatty acids, such as eicosapentaenoic acid, docosahexaneoic acid, docosapentaenoic acid, and esters thereof, to treat or prevent the symptoms of pulmonary hemorrhage in an animal. In one embodiment, the feed composition is supplemented with an omega-3 fatty acid-containing composition comprising DHA and EPA, wherein the DHA:EPA ratio in the feed composition as a final mixture is about 3:1 to about 1:3.

A biologically effective amount of the omega-3 fatty acid-containing composition can be administered to treat or prevent the symptoms of pulmonary hemorrhage in an animal. By “biologically effective amount” is meant an amount of the omega-3 fatty acid-containing composition capable of treating or preventing the symptoms of pulmonary hemorrhage in an animal by any mechanism, including those described herein. Additionally, a biologically effective amount of the omega-3 fatty acid-containing composition can be an amount capable of decreasing the red blood cell count in the airways of an animal.

As described herein, “treating pulmonary hemorrhage” means to decrease the symptoms of pulmonary hemorrhage in an animal known to suffer from pulmonary hemorrhage, for example, to improve or eliminate the existing symptoms of pulmonary hemorrhage.

As described herein, “preventing pulmonary hemorrhage” means preventing the symptoms of pulmonary hemorrhage in an animal not presently suffering from the symptoms of pulmonary hemorrhage.

The compositions and methods as herein described are useful in the method of treating or preventing pulmonary hemorrhage in an equine species, for example, horses, ponies, donkeys, mules, and the like. More particularly, the composition is useful in treating or preventing pulmonary hemorrhage in horses, including racehorses, jumpers, polo ponies, draft horses, and the like. The compositions and methods described herein are also useful in treating or preventing pulmonary hemorrhage in dogs such as greyhounds used for racing and in animals such as camels used for racing.

As described herein, pulmonary hemorrhage may include exercise induced pulmonary hemorrhage, occurring after exposure of the animal to exercise, or any type of physical activity or exertion. In one embodiment, symptoms of pulmonary hemorrhage include, but are not limited to, the presence of red blood cells in the airways of the animal, bleeding from the nostrils (epistaxis), coughing, frequent swallowing, and dyspnea. In other embodiments, symptoms may include engorgement of the pulmonary arteries, veins, and capillaries with hemorrhage into alveoli, bronchioles, bronchi, interstitium, and subpleural tissues.

The presence of red blood cells in the airways of an animal may be determined through any art recognized technique, including examination of bronchoalveolar lavage fluid (obtained by bronchoalveolar lavage, a diagnostic technique in which fluid is instilled into the lungs and removed for examination), endoscopic examination, etc.

In one embodiment, the feed compositions that contain omega-3 fatty acids are administered to the animal orally, but any other effective method of administration known to those skilled in the art may be utilized. In one illustrative embodiment, the feed composition is administered daily to the animal. In one illustrative aspect, the feed composition as a final mixture may be administered to the animal for any time period that is effective to treat or prevent the symptoms of pulmonary hemorrhage in an animal. For example, the feed composition may be fed to the animal daily for the lifetime of the animal. Alternatively, the feed composition may be administered to the animal for a shorter time period. For example, desired results are observed after 30 days, 60 days, 90 days, 120 days, and 150 days or more of administrating the feed composition to the animal.

In illustrative embodiments, as a source of omega-3 fatty acids, the feed composition as a final mixture may comprise products from an algal source (e.g., algal oils, dried algal products, and residuals and derivatives thereof), fish sources (e.g., fish oils, fish meal products, and oil derived from fish meal), or a nut, seed, or plant derived product (e.g., walnut, flaxseed, canola, soybean oil, or corn oil, or a derivative thereof), or combinations thereof. In illustrative embodiments, the feed composition as a final mixture may be supplemented with any omega-3 fatty acid-containing composition, and may include, for example, an algal oil, a dried algal product (including dried whole cells and ground algal products), a fish oil (e.g., menhaden oil, haddock oil, salmon oil or another fish oil from a North Atlantic cold water fish), fish meal, or an oil derived from fish meal, or a mixture thereof, or residuals from any of these sources of omega-3 fatty acids to provide a source of omega-3 fatty acids/esters in a mixture with an art-recognized animal feed blend.

As described herein, “fish meal” is derived from ground dried fish or fish waste, including dried, ground tissue of whole fish or fish cuttings, with or without the extraction of part of the oil.

As described herein, “fish oil” is the oil derived from the tissues of fish and/or fish byproducts. Fish oil, as used herein, may include oil derived from a fish meal product. However, fish oil, whether derived from a fish meal product or from tissues of fish and/or fish byproducts, is not equivalent to fish meal.

In one embodiment, the feed composition as a final mixture can be supplemented with an omega-3 fatty acid-containing composition derived from algae, such as oils, gels, pastes, dried products, and derivatives thereof. In other embodiments, the omega-3 fatty acid-containing composition may include whole algal cell products, ground algal products, or residual products remaining from the production of oils, gels, pastes, and dried products, or derivatives thereof. In illustrative aspects, the algal product may be obtained from any algal source, including marine or freshwater algal sources.

In another embodiment, the animal feed blend is supplemented with an omega-3 fatty acid-containing composition derived from a fish source, such as fish oils or fish meal, as well as plant, nut, or seed oils, or a derivative thereof, or a combination thereof. The omega-3 fatty acid-containing composition derived from a fish source may also include compositions derived from a genetically modified organism. The fish oils described herein may be obtained from any source. In one embodiment, the fish oil source is North Atlantic cold water fish. Fish oils obtained from North Atlantic cold water fish for use in accordance with the present invention include salmon oil, menhaden oil, haddock oil, mackerel oil, herring oil, and the like, but fish oils from sources other than North Atlantic cold water fish may also be used in accordance with the present invention.

Fish oils provide a source of both omega-3 and omega-6 fatty acids, but are a particularly good source of omega-3 polyunsaturated fatty acids. The omega-3 polyunsaturated long chain fatty acids eicosapentaenoic acid (20:5w3), docosahexaneoic (22:6w3), and docosapentaenoic acid (22:5w3) are typical of fish oils and together comprise about 25-38% by weight of the fish oil. Omega-6 polyunsaturated fatty acids present in fish oils include linoleic acid and arachidonic acid and are present at lesser concentrations of about 10% by weight.

Oils are understood to be lipids or fats including the glyceride esters of fatty acids along with associated phosphatides, sterols, alcohols, hydrocarbons, ketones, alkyl esters, salts, and related compounds. Further, as described herein, dried products include algal and non-algal products prepared by any method known in the art, and may include spray-dried or freeze-dried products. The omega-3 fatty acid containing products described herein may include whole cell products, ground products, or derivatives thereof.

In various illustrative aspects, the oils or fatty acid ester components may be added in an unprocessed form or in pure form, and may be conjugated or unconjugated. Illustratively, the fatty acid esters added to the feed composition may be in the form of triglycerides, diglycerides, monoglycerides, phospholipids, lysopholipids, or can be chemically beneficiated or enzymatically beneficiated for enhanced content of desired fatty acid esters.

In various illustrative embodiments, any animal feed blend known in the art may be used to make the feed composition such as rapeseed meal, flaxseed meal, cottonseed meal, soybean meal, and cornmeal. The animal feed blend can be supplemented with an omega-3 fatty acid-containing composition, but other ingredients may optionally be added to the animal feed blend. Optional ingredients of the animal feed blend include sugars and complex carbohydrates such as both water-soluble and water-insoluble monosaccharides, disaccharides and polysaccharides. Optional amino acid ingredients that may be added to the feed blend are arginine, histidine, isoleucine, leucine, lysine, methionine, phenylalanine, threonine, tryptophan, valine, tyrosine ethyl HCl, alanine, aspartic acid, sodium glutamate, glycine, proline, serine, cysteine ethyl HCl, and analogs, and salts thereof. Vitamins that may be optionally added are thiamine HCl, riboflavin, pyridoxine HCl, niacin, niacinamide, inositol, choline chloride, calcium pantothenate, biotin, folic acid, ascorbic acid, and vitamins A, B, K, D, E, and the like. Optional lipid blends of animal or plant origin or fiberous ingredients could also be added. Protein ingredients may also be added and include protein obtained from meat meal or fish meal, liquid or powdered egg, fish solubles, and the like. Any medicament ingredients known in the art may also be added to the animal feed blend, for example, antibiotics may be added.

In an illustrative embodiment, antioxidants may be added to the feed composition to prevent oxidation of the fatty acids present in the omega-3 fatty acid-containing composition used to supplement the feed composition, such as the omega-3 long chain fatty acids, eicosapentaenoic acid, docosahexaneoic acid, and docosapentaenoic acid. Oxidation of fatty acids occurs over time and may be affected by such conditions as moisture and the presence of mineral catalysts and by such characteristics of fatty acids as the number of double bonds and positioning and configuration of bonds. Oxidation of these omega-3 fatty acids can be prevented by the introduction of naturally-occurring antioxidants, such as beta-carotene, vitamin E, vitamin C, and tocopherol or of synthetic antioxidants such as butylated hydroxytoluene, butylated hydroxyanisole, tertiary-butylhydroquinone, propyl gallate or ethoxyquin to the feed composition. Compounds which act synergistically with antioxidants can also be added such as ascorbic acid, citric acid, and phosphoric acid. The amount of antioxidants incorporated in this manner depends on requirements such as product formulation, shipping conditions (e.g., shipping under a nitrogen blanket), packaging methods, and desired shelf life.

In one embodiment, the feed compositions described herein may also comprise omega-6 fatty acids or esters thereof, as described in U.S. Pat. No. 7,084,175 and U.S. patent application Ser. No. 10/142,685, incorporated herein by reference. Illustratively, the omega-6 fatty acids usable in the present invention can be unsaturated fatty acids having at least two carbon-carbon double bonds such as 2,4-decadienoic acid, linolenic acid, gamma-linolenic acid, 8,10,12-octadecatrienoic acid and arachidonic acid. In another embodiment, the omega-6 fatty acid can be gamma-linolenic acid. In other embodiments, omega-6 fatty acids/esters for use in the feed composition of the present invention include omega-6 fatty acids/esters derived from an art-recognized meal such as corn meal or soybean meal or from oils such as corn oil, cottonseed oil, soybean oil, safflower oil, sunflower oil, linseed oil, borage oil, blackcurrant oil, evening primrose oil, and the like.

In one illustrative aspect, the feed composition described herein is supplemented with concentrations of an omega-3 fatty acid-containing composition, such as algal oil, gel, paste, dried products, or a combination thereof, or residuals thereof, sufficient to provide amounts of omega-3 fatty acids/esters in the feed composition as a final mixture that are effective in treating or preventing pulmonary hemorrhage in an animal. Alternatively, the feed composition may be supplemented with an omega-3 fatty acid-containing composition, such as fish oil, fish meal, plant-derived products, or combinations thereof, sufficient to provide amounts of omega-3 fatty acids/esters in the feed composition as a final mixture that are effective in treating or preventing pulmonary hemorrhage in an animal. In another embodiment, the feed composition may be supplemented with a combination of any of the above omega-3 fatty acid-containing sources.

In various aspects, the omega-3 fatty acid-containing composition as described herein may be administered in an unencapsulated or an encapsulated form in a mixture with an animal feed blend. Encapsulation protects the omega-3 fatty acids/esters and omega-6 fatty acids/esters from breakdown and/or oxidation prior to digestion and absorption of the fatty acids/esters by the animal (i.e., encapsulation increases the stability of fatty acids) and provides a dry product for easier mixing with an animal feed blend. The omega-3 fatty acids/esters and omega-6 fatty acids/esters can be protected in this manner, for example, by coating the oil with a protein or any other substances known in the art to be effective encapsulating agents such as polymers, waxes, fats, and hydrogenated vegetable oils. For example, an oil or other algal or fish product, may be encapsulated using an art-recognized technique such as a Na²⁺-alginate encapsulation technique wherein the oil is coated with Na²⁺-alginate followed by conversion to Ca²⁺-alginate in the presence of Ca²⁺ions for encapsulation. Alternatively, the oil or other algal or fish product may be encapsulated by an art-recognized technique such as enrobing the fatty acids to stabilize the fatty acids or prilling (i.e., atomizing a molten liquid and cooling the droplets to form a bead). For example, the oil or other algal or fish product may be prilled in hydrogenated cottonseed flakes or hydrogenated soy bean oil to produce a dry oil. In various embodiments, the oil or other algal or fish product may be used in an entirely unencapsulated form, an entirely encapsulated form, or mixtures of unencapsulated and encapsulated oil may be added to the feed composition.

While certain embodiments of the present invention have been described and/or exemplified below, it is contemplated that considerable variation and modification thereof are possible. Accordingly, the present invention is not limited to the particular embodiments described and/or exemplified herein.

### Example 1

### Animals and Experimental Design

Thoroughbred horses (approximately 4 to 10 years of age) were used to study the effect of daily omega-3 fatty acid supplementation on measures of exercise-induced pulmonary Hemorrhage (EIPH). Horses were transported and treated two per pen in outdoor paddocks. Horses were blocked by body weight and approximate age and assigned randomly within each block to two dietary treatments. Dietary treatments included (1) feed supplemented with omega-3 carrier only, or (2) concentrate feed supplemented with omega-3 supplement. Both dietary feed treatments were top-dressed onto the grain-based supplement provided in two equal feedings daily. Horses were also fed grass and alfalfa hay twice daily. Hay and concentrate were fed to meet or exceed the NRC requirements of horses at moderate to heavy work. Horses were provided ad libitum access to water and salt licks. The dietary treatments were administered for a period of 145 days. Horses were fed approximately 20 grams/day of DHA and approximately 10 grams/day of EPA.

All horses were dewormed with ivermectin and vaccinated against eastern and western encephalomyelitis, tetanus, equine influenza, West Nile virus, and equine herpes I, prior to beginning the dietary treatment. Beginning two weeks prior to initiation of the study, and continuing throughout the course of the study, horses were trained on a high-speed treadmill three days/week, on a moderate-severe intensity exercise regimen (≦10 m/s on flat; ≦7 m/s on inclined treadmill). Feed was withheld for at least two hours before each training session.

### Example 2

### Lavage Fluid Red Blood Cell Assay

A bronchoalveolar lavage (BAL) tube was placed in a designated container containing 70% isopropyl alcohol, well in advance of running the horse. A syringe (20 cc) was used to flush alcohol through the BAL tube (through stopcock). The tube was left in alcohol until time for lavage (at least 10-15 minutes). The BAL tube was then removed and flushed inside while rinsing outside with 3-60 cc syringes of saline followed by 60 cc of air. The horse was then restrained with a halter and leadrope (±a twitch), sedated with either xylazine hydrochloride (Rompin) at a dose of 0.5-1 mg/kg i.v. or detomidine hydrochloride (Dormosedan) at a dose of 0.01-0.03 mg/kg i.v. (can be combined with butorphanol tartrate (Torbugesic) at a dose of 0.01-0.03 mg/kg i.v.). In addition, a local anesthetic was administered (e.g., lidocaine (20 ml of a 2% solution diluted with saline to a total volume of 100 ml and administered at the carina (bifurcation of the trachea)) to suppress coughing.

To pass the tube, the left hand was cupped around the bridge of the horse's nose without occluding the nostrils. The tube was inserted into the nostril, staying ventrally to avoid the false nostril. The tube was advanced to the arytenoid cartilages. When the horse took a breath the tube was advanced into the trachea. The location in the trachea was confirmed because the horse often coughed upon entering the trachea and at the bifurcation of the lungs. Also, the tube had no resistance upon advancement and air came through the end of the tube held in the hand. The tube was passed until gentle resistance was felt indicating wedging of the tube in a sub-segmental bronchus of the dorsocaudal lung. At this point a 3 cc syringe full of air was inserted into the cuff to maintain the location of the tube in the lung.

Next, 300 ml of warmed saline (0.9%) was instilled into the lung through the BAL channel in either 5, 60 cc increments or 6, 50 cc increments, one immediately followed by the next until the total volume was instilled. After the horse took a couple of breaths, the fluid was aspirated into 60 cc syringes with gentle aspiration until negative pressure was obtained (typical recoveries contain 50-60% of instilled fluid). The recovered fluid was securely capped, making sure that no leaks occurred when the syringe was turned upside down and immediately placed on ice. If less than 100 ml total volume minus 17 ml dead space was recovered, 150-300 ml could be reinstilled, leaving the catheter in place, and repeating instillation and aspiration procedures. Once the procedure was completed, the cuff was deflated and the BAL tube was slowly removed as to avoid bumping the ethmoids and causing a nose bleed.

The conical tubes were then spun in the centrifuge at 2300 rpm for 10 minutes. A total volume of recovery fluid (meniscus on conical tube was read and all tubes were added to give total volume) was recorded. A vacuum pipette was used to remove surfactant and fluid down to the pellet without disturbing the pellet. The cells were resuspended in 0.9% saline (normalizing the number of red blood cells by making the fluid look the same color each time, (e.g., a cherry Koolade color). The “resuspension” volume was recorded. The sample was diluted in some cases 1:10 (1 ml sample+9 mls saline) to count both RBCs and WBCs in the same dilution. 20 μl sample+20 μl trypan blue stain (for viability) was mixed in small centrifuge tubes (pipette 10-15×) and loaded into hemocytometer chambers. The red blood cells and white blood cells were counted by methods known in the art. The blood cells can also be counted by employing flow cytometry, using standard methods known in the art.

### Example 3

### Lavage Fluid Red Blood Cell Data

Prior to initiation of the dietary treatments, and at 83 days and 145 days of feeding each dietary treatment, horses were subjected to a maximal exercise run including an incremental exercise test [warm-up (3 m/s for 800 m); step test (1 m/s per min. increments to fatigue); and recovery (3 m/s for 800 m)] on an inclined treadmill at 10% grade. Heart rate was obtained from a Polar Heart Rate monitor at each stage of the exercise test (rest, exercise, and recovery). Immediately after the conclusion of the test, jugular samples of blood were obtained. Additionally, the horses were tranquilized for bronchoalveolar lavage (BAL).

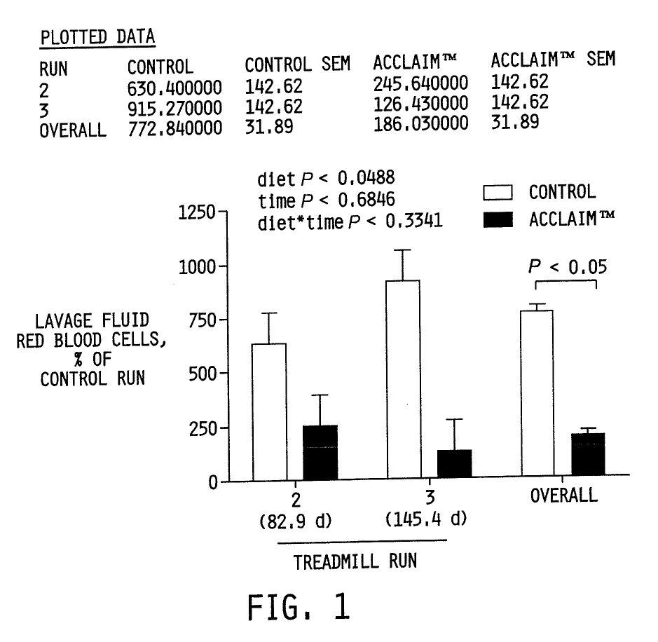

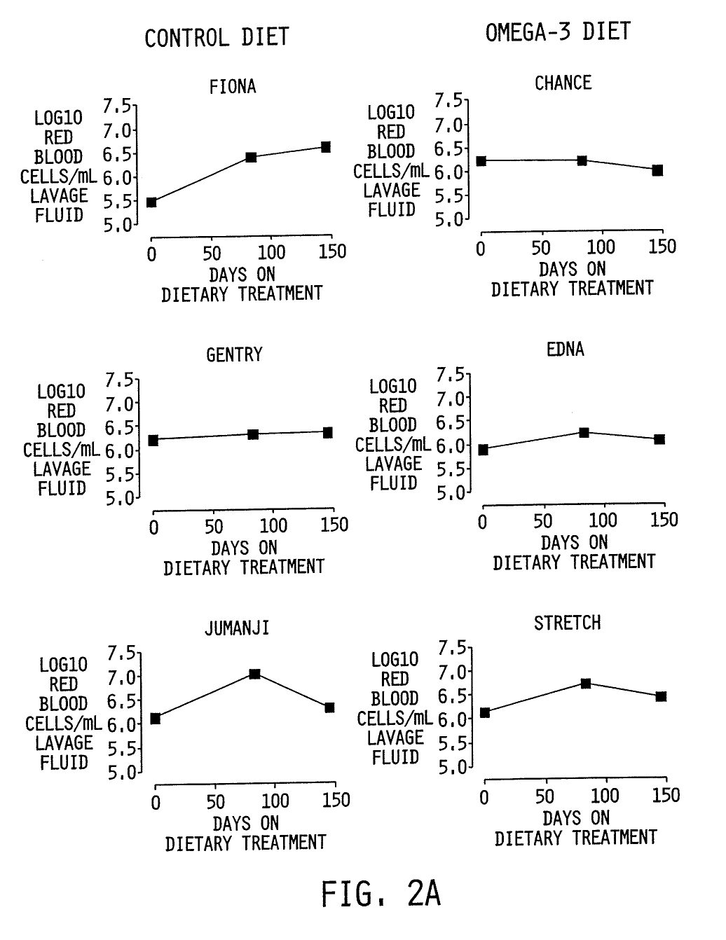

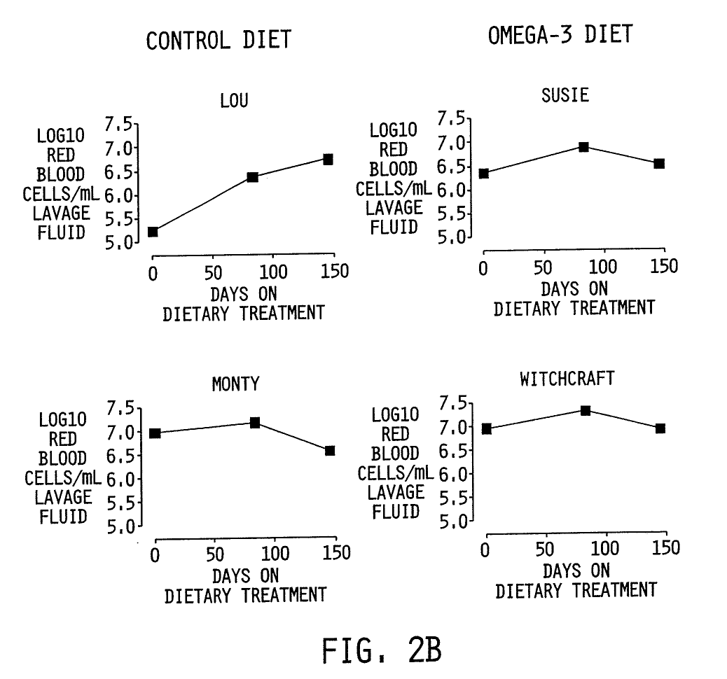

Lavage fluid red blood cell (RBC) data was expressed as a percentage of prefeeding baseline Max Run 1 (see FIGS. 1 and 2). The RBC counts in lavage fluid obtained after Max Runs 2 and 3, when expressed as a percentage of the RBC count following baseline Max Run 1, were lower for horses fed the diets supplemented with omega-3 fatty acids (P<0.05). In the control horses, RBC counts increased in the second and third runs relative to the baseline run. The raw data suggests that the greatest increase among control horses was in those horses that were relatively light bleeders in the initial baseline run (notably Fiona and Lou, and to a lesser extent Jumanji (see FIG. 2), who recovered somewhat at the third run).

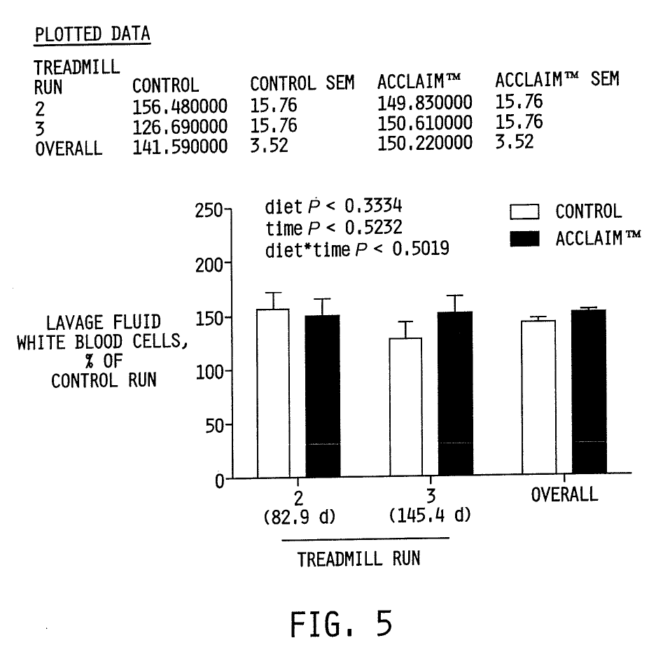

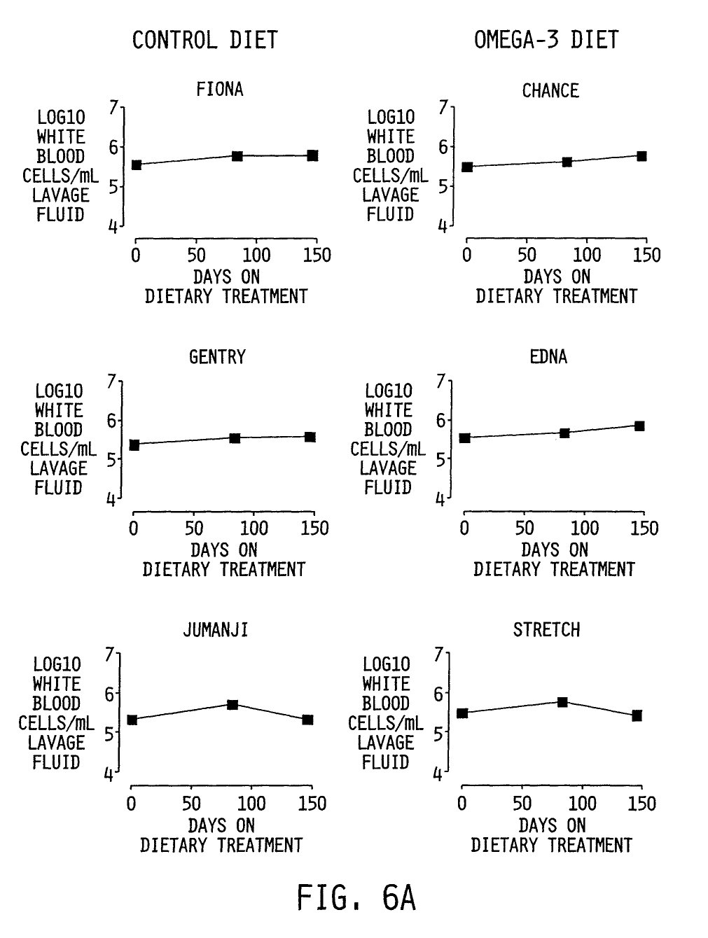

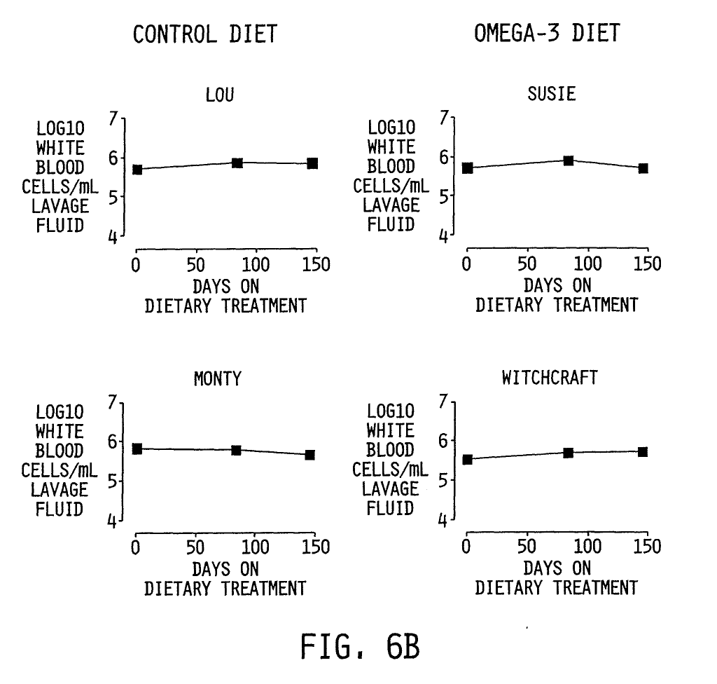

Horses fed the omega-3 supplement had RBC counts that were 245.6% and 126.4% of their control runs at Max Runs 2 and 3, respectively. However, these changes were much lower than those observed in control horses (see FIG. 1). The data suggest that the diet supplemented with omega-3 fatty acids is beneficial in the management of established EIPH in horses. Additionally, the omega-3 supplemented diet failed to affect lavage white blood cell (WBC) counts (FIGS. 5 and 6), suggesting that changes in RRCs in response to dietary treatment were independent of changes in WBCs.

### Example 4

### Serum Eicosapentaenoic Acid, Docosahexaenoic Acid, Alpha Linolenic Acid, and Arachidonic Acid

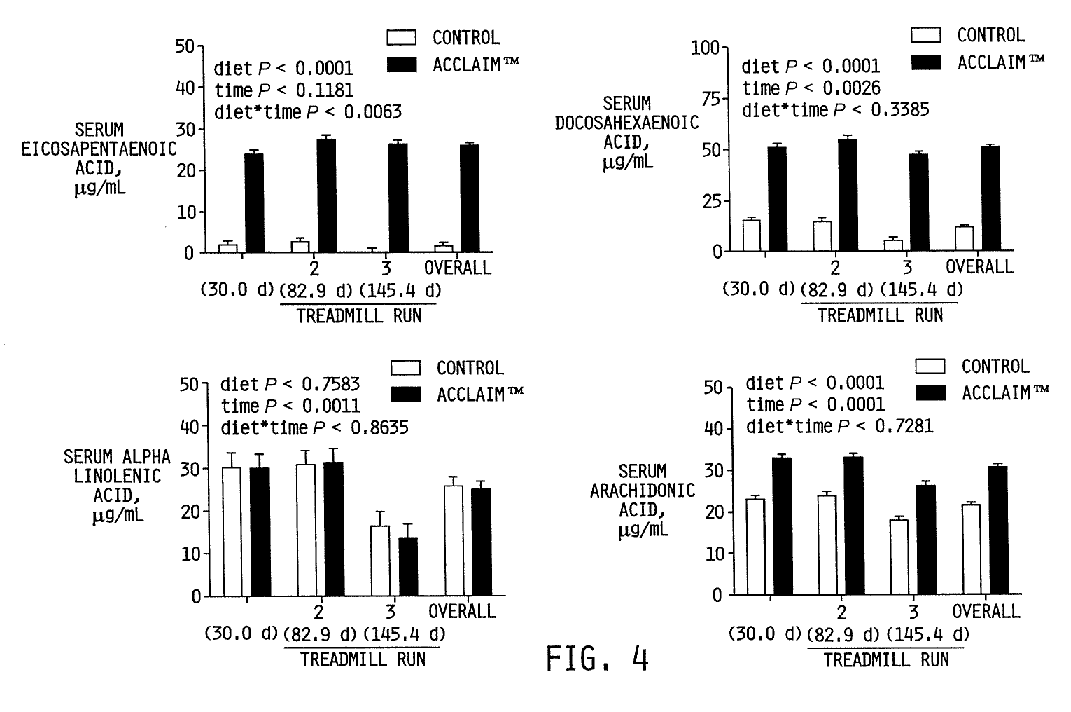

Serum fatty acids were quantified in three samples obtained after 30 days of feeding and at treadmill runs at 83 days and 145 days. Serum long chain fatty acids, including serum eicosapentaenoic acid (EPA), docosahexaenoic acid (DHA), alpha linolenic acid (ALA), and arachidonic acid (ARA), are illustrated in FIG. 4. Serum concentrations of EPA, DHA, and ARA were elevated in horses fed the omega-3 enriched diet. The elevations, particularly in serum EPA and DHA, are clear and confirm the effectiveness of the dietary enrichment of EPA and DHA in the grain portion of the diet.

Also shown is a treatment by time interaction (P<0.01) for serum EPA. Whereas serum EPA was markedly elevated above that of control horses at each sampling time (P<0.0001), the interaction may be due to the drop in EPA in control horses at the final sampling time. In fact, serum EPA in all control horses was below the limit of detection of the assay in those samples. The most likely source of the small amount of circulating EPA in control horses was from the hay component of their diet, and this change may reflect poorer quality hay fed between the second and third sampling times. Likewise, serum DHA was also lower in this sample compared to samples collected earlier in the study. The treatment effect on serum ARA was not anticipated. Although it was of lower magnitude than either serum omega-3 fatty acid, the consistent elevation in ARA suggests that it was, in fact, associated with feeding the omega-3 enriched diet.

### Example 5

### Lavage Phagocytosis-Oxidative Burst

BAL fluid was used to determine the severity of EIPH. The measurements included counts of red blood cells, mast cells (specific staining with toluidine blue), and total nucleated cells (TNCs) using a hemocytometer, as well as differential white blood cell counts. Both BAL fluid and peripheral blood mononuclear cells (from jugular blood) were used to determine PGE₂ response to LPS. Neutrophil phagocytic-oxidative burst function was determined using flow cytometry.

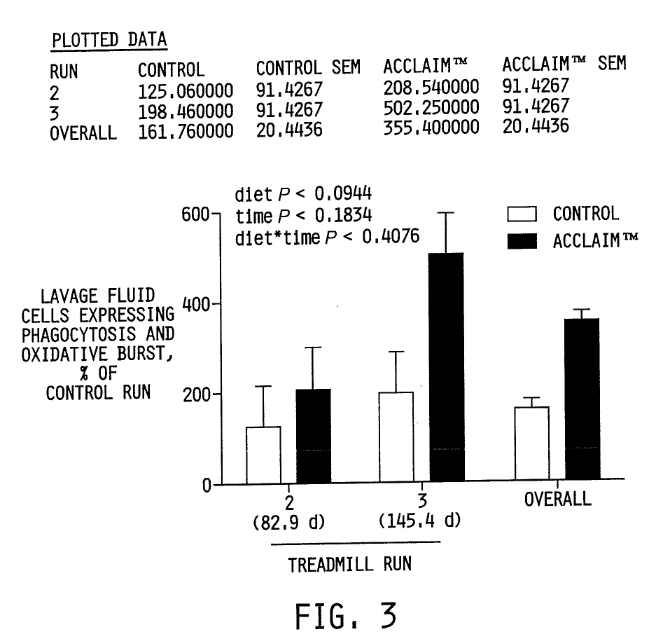

Phagocytosis-oxidative burst functions of inflammatory cells in lavage fluid increased in horses fed the diet enriched in omega-3 fatty acids, particularly at Max Run 3 (FIG. 3 and Table 1).

TABLE 1

| | | | | | % | % |
|---|---|---|---|---|---|---|
| Horse | | PO1 | PO2 | PO3 | Change | Change |
| CHANCE | Omega3 | 20.8 | 28.24 | 20.94 | 135.77 | 100.67 |
| WITCH | Omega3 | 11.82 | 50.3 | 44.68 | 425.55 | 378.00 |
| SUSIE | Omega3 | 21.06 | 11.3 | 57.1 | 53.66 | 271.13 |
| STRETCH | Omega3 | 23.32 | 7.3 | 23.04 | 31.30 | 98.80 |
| EDNA | Omega3 | 2.81 | 11.14 | 46.72 | 396.44 | 1662.63 |
| JUMANJI | Control | 8.54 | 23.23 | 29.69 | 272.01 | 347.66 |
| FIONA | Control | 13.05 | 16.71 | 49.2 | 128.05 | 377.01 |
| MONTY | Control | 33.54 | 31.5 | 40.17 | 93.92 | 119.77 |
| GENTRY | Control | 21.14 | 23.68 | 19.27 | 112.02 | 91.15 |
| LOU | Control | 20.82 | 4.02 | 11.81 | 19.31 | 56.72 |

### Example 6

### Feed Compositions

Animals were fed dietary treatments, including (1) feed not supplemented with omega-3 fatty acids, or (2) feed supplemented with omega-3 supplement. The diets fed to the animals were pelleted diets, however, the formulation can also be extruded or provided in a meal form, or any other art recognized form. The diets as used herein are shown in Table 2 and Table 3.

TABLE 2
Equine Control Diet

| Ingredient | lbs/Ton | % |
|---|---|---|
| Corn | 766 | 38.3 |
| Oat-Ground | 500 | 25 |
| SBM-48% | 284 | 14.2 |
| Alfalfa | 239.4 | 11.97 |
| Molasses-Dry | 100 | 5 |
| Choice White Grease | 61.8 | 3.09 |
| Mono-Cal | 26.8 | 1.34 |
| Vitamin E 20 | 16 | 0.8 |
| Horse VTM | 6 | 0.3 |
| | 2000 | 100 |

TABLE 3
Equine FA Diet

| Ingredient | lbs/Ton | % |
|---|---|---|
| Corn | 743 | 37.15 |
| Oat-Ground | 500 | 25 |
| SBM-48% | 302.2 | 15.11 |
| Alfalfa Meal | 200 | 10 |
| Molasses-Dry | 100 | 5 |
| Menhaden Oil | 70.5 | 3.525 |
| DHA Gold ™* | 35.3 | 1.765 |
| Mono-Cal | 27 | 1.35 |
| Vitamin E 20 | 16 | 0.8 |
| Horse VTM | 6 | 0.3 |
| | 2000 | 100 |

*S-Type Gold Fat, DHA Gold ™; Advanced BioNutrition, Columbia, MD).

What is claimed is:

1. A method of preventing pulmonary hemorrhage in an animal, said method comprising the step of administering to said animal an effective amount of a feed composition comprising omega-3 fatty acids or esters thereof to prevent pulmonary hemorrhage in said animal, wherein the omega-3 fatty acids in the final feed composition comprise docosahexaenoic acid and eicosapentaenoic acid, and wherein the docosahexaenoic acid to eicosapentaenoic acid ratio in the final feed composition is about 3:1 to about 1:3.

2. The method of claim 1 wherein the source of omega-3 fatty acids or esters thereof is selected from the group consisting of an algal composition, a fish composition, and combinations thereof.

3. The method of claim 2 wherein the algal composition is in the form of algal products selected from the group consisting of algal-derived oils, algal-derived gels, algal-derived pastes, algal-derived dried products, and derivatives thereof.

4. The method of claim 2 wherein the algal composition comprises algal products selected from the group consisting of whole algal cell products, ground algal products, and residual products.

5. The method of claim 2 wherein the fish composition is selected from the group consisting of a fish oil, a fish meal product, and combinations thereof.

6. (canceled)

7. The method of claim 5 wherein the fish oil comprises an oil selected from the group consisting of menhaden oil, salmon oil, and haddock oil.

8.-12. (canceled)

13. The method of claim 1 wherein the docosahexaenoic acid to eicosapentaenoic acid ratio in the final feed composition is about 2:1.

14. The method of claim 1 wherein the amount of the docosahexaenoic acid fed to said animal is at least 10 grams/day.

15. (canceled)

16. The method of claim 1 wherein the pulmonary hemorrhage is exercise-induced pulmonary hemorrhage.

17. (canceled)

18. A method of treating pulmonary hemorrhage in an animal, said method comprising the step of administering to said animal an effective amount of a feed composition comprising omega-3 fatty acids or esters thereof to treat pulmonary hemorrhage in said animal, wherein the omega-3 fatty acids in the final feed composition comprise docosahexaenoic acid and eicosapentaenoic acid, and wherein the docosahexaenoic acid to eicosapentaenoic acid ratio in the final feed composition is about 3:1 to about 1:3.

19. The method of claim 18 wherein the source of omega-3 fatty acids or esters thereof is selected from the group consisting of an algal composition, a fish composition, and combinations thereof.

20. The method of claim 19 wherein the algal composition is in the form of algal products selected from the group consisting of algal-derived oils, algal-derived gels, algal-derived pastes, algal-derived dried products, and derivatives thereof.

21. The method of claim 19 wherein the algal composition comprises algal products selected from the group consisting of whole algal cell products, ground algal products, and residual products.

22. The method of claim 19 wherein the fish composition is selected from the group consisting of a fish oil, a fish meal product, and combinations thereof.

23. (canceled)

24. The method of claim 22 wherein the fish oil is selected from the group consisting of menhaden oil, salmon oil, and haddock oil.

25.-29. (canceled)

30. The method of claim 18 wherein the docosahexaenoic acid to eicosapentaenoic acid ratio in the final feed composition is about 2:1.

31. The method of claim 18 wherein the amount of the docosahexaenoic acid fed to said animal is at least 10 grams/day.

32. (canceled)

33. The method of claim 18 wherein the pulmonary hemorrhage is exercise-induced pulmonary hemorrhage.

34. (canceled)

35. A method of decreasing the red blood cell count in the airways of an animal, said method comprising the step of administering to said animal an effective amount of a feed composition comprising omega-3 fatty acids or esters thereof to decrease the red blood cell count in the airways of said animal, wherein the omega-3 fatty acids in the final feed composition comprise docosahexaenoic acid and eicosapentaenoic acid, and wherein the docosahexaenoic acid to eicosapentaenoic acid ratio in the final feed composition is about 3:1 to about 1:3.

36. The method of claim 35 wherein the source of omega-3 fatty acids or esters thereof is selected from the group consisting of an algal composition, a fish composition, and combinations thereof.

37. The method of claim 36 wherein the algal composition is in the form of algal products selected from the group consisting of algal-derived oils, algal-derived gels, algal-derived pastes, algal-derived dried products, and derivatives thereof.

38. The method of claim 36 wherein the algal composition comprises algal products selected from the group consisting of whole algal cell products, ground algal products, and residual products.

39. The method of claim 36 wherein the fish composition is selected from the group consisting of a fish oil, a fish meal product, and combinations thereof.

40. (canceled)

41. The method of claim 39 wherein the fish oil comprises an oil selected from the group consisting of menhaden oil, salmon oil, and haddock oil.

42.-46. (canceled)

47. The method of claim 35 wherein the docosahexaenoic acid to eicosapentaenoic acid ratio in the final feed composition is about 2:1.

48. The method of claim 35 wherein the amount of the docosahexaenoic acid fed to said animal is at least 10 grams/day.

49. (canceled)

50. The method of claim 35 wherein the pulmonary hemorrhage is exercise-induced pulmonary hemorrhage.

51. (canceled)
